(19) United States
(12) Patent Application Publication
(10) Pub. No.: US 2016/0130561 A1
(43) Pub. Date: May 12, 2016

(54) PLURIPOTENT STEM CELLS DERIVED FROM NON-CRYOPROTECTED FROZEN TISSUE AND METHODS FOR MAKING AND USING THE SAME

(71) Applicants: The New York Stem Cell Foundation Inc., New York, NY (US); The Trustees of Columbia University in the City of New York, New York, NY (US)

(72) Inventors: Andrew Sproul, New York, NY (US); John Crary, Brooklyn, NY (US); Lauren Ab Vensand, New York, NY (US); Carmen Dusenberry, Bronx, NY (US); Scott Noggle, Long Island City, NY (US); Michael Shelanski, Brooklyn, NY (US)

(21) Appl. No.: 14/897,225

(22) PCT Filed: Jun. 13, 2014
(86) PCT No.: PCT/US14/42398

§ 371 Date: Dec. 9, 2015

Related U.S. Application Data
(60) Provisional application No. 61/834,745, filed on Jun. 13, 2013.

(51) Int. Cl. C12N 5/074; G01N 33/50

(52) U.S. Cl. CPC C12N 5/0696; G01N 33/5073

(57) ABSTRACT

In some embodiments the present invention provides cells, such as pluripotent stem cells and differentiated cells, derived from tissue samples that have been frozen without a cryoprotective agent, and also cell panels comprising such cells, model systems comprising such cells, and methods for making and using such cells, cell panels, and model systems.

### CROSS-REFERENCE TO RELATED APPLICATIONS

This application claims the benefit of priority of U.S. provisional patent application 61/834,745 filed on Jun. 13, 2013, the contents of which are hereby incorporated by reference in their entirety.

### STATEMENT OF GOVERNMENT RIGHTS

This invention was made with government support under grant number 1RF1AG042965-02 awarded by the National Institutes of Health/National Institute on Aging, and grant number P30AG036453 awarded under The American Recovery and Reinvestment Act (ARRA). The government has certain rights in the invention.

### COPYRIGHT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

The text of all documents cited herein is hereby incorporated by reference in its entirety.

### BACKGROUND

Pluripotent stem cells, for example induced pluripotent stem cells (iPSCs), have been used to develop in vitro models of diseases and to conduct screening. However, for some diseases, tissue from patients with similar symptoms or diagnoses exhibit different pathology, and some diseases may not be correctly or definitely diagnosed until post-mortem tissue analysis.

While generation of iPSCs from fresh autopsy tissue has recently been reported (Bliss L A et al. PLoS ONE 2012, 7:e45282; Hjelm B E et al. Neurosci Lett 2011, 502:219-224), tissue banks and biorepositories contain tens of thousands of well-characterized samples and could provide a large, immediate source of tissue for development of pluripotent stem cell-based model systems for a variety of diseases and disorders. Archived collections represent a diversity of diseases and disorders, including neurodegenerative diseases, HIV/AIDS, and neoplasia, many of which currently have a lack of cellular models. However, the majority of biobanked samples were not meant for growing live cells, and thus were not frozen in the presence of cryoprotectants, such as DMSO, which can protect tissues and cells from damage related to freezing. Accordingly, such archived and biobanked tissues have not generally be considered to be of use for the generation of living cells and for the generation of cellular model systems. There is a need in the art for methods that can unlock the potential of non-cryoprotected samples to support the development of personalized medicine and novel therapeutic agents, and to further translational research and associated genetic studies.

### SUMMARY OF THE INVENTION

Some of the main aspects of the present invention are summarized below. Additional aspects are described in the Detailed Description of the Invention, Drawings, Claims and Examples sections of the application.

In some embodiments, the present invention provides an in vitro model system for studying cells, or for studying the effect of one or more agents on cells, the model system comprising (a) a cell panel comprising at least two cell populations, wherein each cell population (i) was derived from a tissue sample obtained from a subject, wherein the tissue sample had been frozen without a cryoprotective agent, and (ii) comprises pluripotent stem cells or differentiated cells derived following reprogramming of differentiated somatic cells grown from the tissue sample; and (b) a data panel comprising information associated with each cell population in the cell panel or each tissue sample or subject from which each cell population was derived.

In some embodiments, the present invention provides a model system for a disease or disorder, the model system comprising (a) a panel of cells (for example, somatic cells, multipotent cells, pluripotent stem cells, induced pluripotent stem cells, differentiated cells or neurons) comprising at least two populations of cells, wherein each population of cells was derived from a tissue sample, wherein the tissue sample had been frozen without a cryoprotective agent, and wherein the tissue sample is from a subject that has or had a disease or disorder; and (b) information obtained from and/or associated with each population of cells of (a) and/or the tissue sample(s) and/or subject(s) of (a) from which the population of cells was derived.

In some embodiments, the invention provides a model system for a disease or disorder, the model system comprising (a) a test or experimental component comprising (i) a panel of cells (for example, somatic cells, multipotent cells, pluripotent stem cells, or differentiated cells such as neurons), comprising at least two populations of cells, wherein each population of cells was derived from a tissue sample, wherein the tissue sample had been frozen without a cryoprotective agent, and wherein the tissue sample is from a subject that has or had a disease or disorder; and (ii) information obtained from and/or associated with the cells of (a)(i) and/or the tissue samples of (a)(i); and (b) a control component comprising (i) a panel of cells (for example, somatic cells, multipotent cells, pluripotent stem cells, or differentiated cells such as neurons), comprising at least two populations of cells, wherein each population of cells was derived from a tissue sample, wherein the tissue sample had been frozen without a cryoprotective agent, and wherein the tissue sample is from a subject that did not or does not have the disease or disorder of (a)(i); and (ii) information obtained from and/or associated with the cells of (b)(i) and/or the tissue samples of (b)(i).

In some embodiments, the present invention provides cell panels for studying cells, or for studying the effect of one or more agents on cells, the cell panels comprising at least two distinct cell populations, wherein each cell population (a) was derived from a tissue sample obtained from a subject, wherein the tissue sample had been frozen without a cryoprotective agent, and (b) comprises pluripotent stem cells or differentiated cells derived following reprogramming of differentiated somatic cells grown from the tissue sample.

In some embodiments, the present invention provides a panel of pluripotent stem cells (for example, induced pluripotent stem cells), the panel comprising at least two populations of pluripotent stem cells, wherein each population of pluripotent stem cells was derived from a tissue sample, and wherein the tissue sample had been frozen without a cryoprotective agent. In some embodiments, the invention provides a panel of differentiated cells (for example, neurons), the panel comprising at least two populations of differentiated cells, wherein each population of differentiated cells was derived from a population of pluripotent stem cells, wherein each population of pluripotent stem cells was derived from a tissue sample, and wherein the tissue sample had been frozen without a cryoprotective agent.

In some embodiments, the invention provides a pluripotent stem cell, an induced pluripotent stem cell, or a differentiated cell (such as a neuron) derived from a tissue sample, wherein the tissue sample had been frozen without a cryoprotective agent. In some such embodiments, a differentiated cell (such as a neuron) may be derived from a pluripotent stem cell or induced pluripotent stem cell derived from the tissue sample. In some embodiments, a differentiated cell may be derived from a pluripotent cell (such as a pluripotent stem cell or an induced pluripotent stem cell) or from another differentiated cell by transdifferentiation.

In some embodiments, the present invention provides a screening method comprising (a) contacting one or more populations of pluripotent stem cells, multipotent cells, or differentiated cells with a candidate agent, wherein each of such population of cells was derived from a tissue sample that had been frozen without a cryoprotective agent, and (b) determining the effect of the candidate agent on the population of cells. In some embodiments, the step of determining the effect of the candidate agent on the population of cells comprises assessing the agent's activity, efficacy, potency, safety, toxicity, or teratogenicity.

In some embodiments, the present invention provides, an in vitro screening method, the method comprising (a) contacting one or more populations of differentiated cells with a candidate therapeutic agent, wherein each population of differentiated cells was derived from a population of pluripotent stem cells, wherein each population of pluripotent stem cells was derived from a tissue sample, wherein the tissue sample had been frozen without a cryoprotective agent, and wherein the tissue sample is from a subject that has or had a disease or disorder; and (b) determining whether the candidate therapeutic agent has a beneficial effect in one or more assays for the disease or disorder. In some embodiments, the assay comprises monitoring one or more indicators of neurodegeneration or neuronal survival.

In some embodiments, the invention provides a method of generating an induced pluripotent stem cell or a differentiated cell, the method comprising (a) obtaining a tissue sample from a subject, wherein the tissue sample had been frozen without a cryoprotective agent, (b) culturing cells from the tissue sample, and (c) contacting the cells cultured in step (b) with one or more reprogramming factors to generate induced pluripotent stem cells or differentiated cells. In some embodiments, the method further comprises contacting the induced pluripotent stem cells of step (c) with one or more differentiation factors to generate differentiated cells.

In some embodiments, the present invention provides a method for generating a population of pluripotent stem cells from a frozen tissue sample, the method comprising (a) obtaining a tissue sample, wherein the tissue sample is frozen, or had previously been frozen, and wherein the tissue sample had been frozen without a cryoprotective agent; (b) culturing cells from the tissue sample in vitro so as to obtain a population of cells from the tissue sample; and (c) deriving a population of pluripotent stem cells from the population of cells of step (b). In some embodiments, the present invention provides a method for generating a population of pluripotent stem cells from frozen dura mater, the method comprising (a) obtaining a dura mater sample, wherein the dura mater sample is frozen, or had previously been frozen; (b) culturing cells from the dura mater sample in vitro so as to obtain a population of cells from the dura mater sample; and (c) deriving a population of pluripotent stem cells from the population of cells of step (b).

In each of the embodiments of the invention the tissue sample may be derived from any suitable or desired subject and may be derived from any suitable or desired tissue from that subject. In some embodiments, the subject is a mammal. In some embodiments, the subject is a human. In some embodiments, the tissue sample may comprise brain tissue, skin tissue, or vascular tissue. In some embodiments, the tissue sample may comprise dura mater tissue. In some embodiments, the tissue sample may comprise a biological fluid, such as blood (such as umbilical cord blood), saliva, or ascites. In some embodiments the tissue sample may be derived from a subject post mortem. In some embodiments, the tissue sample may have been frozen for more than 1 year, more than 5 years, more than 10 years, or more than 20 years. In some embodiments, the tissue sample may have been frozen using liquid nitrogen. In some embodiments, the tissue sample may have been frozen using a liquid nitrogen vapor sandwich method.

In some embodiments of the invention, the tissue sample may have been obtained from a test subject having a certain disease or disorder. In some embodiments the tissue sample may have been obtained from a control subject not having that disease or disorder. The disease or disorder may be any disease or disorder desired—such as any disease or disorder that is desired to be studied, or for which it is desired to screen for the activity of certain candidate agents, such therapeutic agents. For example, in some embodiments, the disease or disorder may be a neurological disease or disorder, including, but not limited to, Alzheimer's disease (AD), Parkinson's disease (PD), amyotrophic lateral sclerosis (ALS), Huntington's disease, multiple systems atrophy (MSA), or diffuse Lewy body disease (DLBD).

In each of the embodiments of the invention, any suitable or desired types of cells can be grown from a tissue sample, such as any suitable type of somatic cells, including, but not limited to fibroblasts (for example, dural fibroblasts) or fibroblast-like cells. Similarly, any suitable or desired type of pluripotent stem cells or differentiated cells can be derived from the tissue sample, or from cells grown from the tissue sample. In some embodiments the pluripotent stem cells may be induced pluripotent stem cells. In embodiments where induced pluripotent stem cells are used, such cells may be derived from differentiated somatic cells obtained from a tissue sample, for example by contacting such differentiated somatic cells with one or more reprogramming factors. In some embodiments the differentiated cells can be any suitable type of differentiated cells. In some embodiments the differentiated cells may be derived from pluripotent stem cells (such as induced pluripotent stem cells), for example by contacting such pluripotent cells with one or more differentiation factors. In some embodiments the differentiated cells may be derived by trans-differentiation of another differentiated cell type (such as a cell type obtained from a tissue sample), for example by contacting the cells with one or more reprogramming factors. In the various embodiments of the present invention involving differentiated cells, such differentiated cells may be any desired differentiated cell type, including, but not limited to, neurons.

In each of the embodiments of the invention the, cells, cell populations, and/or cell panels may be contained in or provided in or on any suitable substrate or vessel. In some embodiments different cell populations are provided in separate vessels, such as wells in a multi-well plate. Also, in each of the embodiments of the invention the, cells, cell populations, and/or cell panels may comprise one or more exogenously-introduced reporter molecules, or exogenously-introduced reprogramming or differentiation factors.

In each of the embodiments of the invention the data panels and/or information may comprise any suitable or desired information. In some embodiments such information may comprise information about a subject's age, sex, race, ethnicity, genotype, phenotype, environment, medical history, health, family history, disease status, treatment history, histopathology, diagnosis, disease classification, disease stratification, disease grading, disease severity, disease progression or any other desired information. In some embodiments, the data panel and/or information may comprise any other information about the subject or the tissue sample or cell population obtained from that subject, such as information relating to genetic analysis, transcriptional analysis, proteomic analysis, or cell culture methods.

These and other aspects of the invention are further described throughout this patent application, including in the drawings, Detailed Description, Examples, and Claims sections of this patent application. Furthermore, one of ordinary skill in the art will recognize that embodiments described herein can be combined and modified in various ways. All such combinations and modifications fall within the scope of the present invention.

### BRIEF DESCRIPTION OF THE FIGURES

FIGS. 1A-1R. Cryoprotected MSA patient outgrowths. Scale bars are 1 cm for (A-B) and 100 μm for (C-L, O-R). (A) Gross examination of a right coronal section through the fresh brain reveals discoloration and atrophy of the globus pallidus and putamen. (B) Examination of the midbrain reveals pallor of the substantia nigra. (C) Luxol-fast blue/hematoxylin and eosin stained sections demonstrate glial cytoplasmic inclusions (Papp-Lantos bodies) in the subcortical white matter. (D) These inclusions are highlighted by immunohisochemical staining to α-synuclein. (E, I) Outgrowths with fibroblast morphology from the scalp (ASC2S-MSA; E) and dura mater (ASC2D-MSA; I). (F-H, J-L) Immunostaining of scalp (ASC2S-MSA-CP) and dura (ASC2D-MSA-CP) iPSCs demonstrates expression of pluripotency markers as indicated. AP stands for alkaline-phosphatase. Nanostring analysis for endogenous stem cell genes (M) and shutoff of Sendai transgenes (N). Hues16 and Hues42 were used as positive controls for endogenous stem cell genes, unrelated fibroblasts as a negative control, and infected unrelated fibroblasts as a positive control for Sendai transgene expression. (O-R) Undirected EBs were cryosectioned and immunostained for the 3 developmental germ layers: endoderm (AFP), mesoderm (SMA), and ectoderm (Tuj1). The presence of endoderm in undifferentiated EBs was also confirmed by positive staining for Sox17 (see U.S. provisional patent application 61/834,745 filed on Jun. 13, 2013, the contents of which are hereby incorporated by reference in their entirety, and to which the present application claims priority).

FIGS. 2A-2N. Generation of iPSCs from non-cryoprotected dura. Scale bars are 1 cm for (A) and 100 μm for (B-M). (A) Gross examination of a coronal right hemi-section from the brain of a sporadic AD patient (case ASCI) illustrates atrophy and ventricular dilatation. Immunohistochemical staining for amyloid-β (Aβ) peptide and hyper-phosphorylated tau confirms pathological accumulation of amyloid plaques and neurofibrillary tangles (insets). Scale bar is 1 cm. (B) Fibroblast-like outgrowths from the thawed archived dura from the same subject, 18 days post plating. (C-N) Characterization of a representative iPSC clone (clone 4) derived ASC7D-AD. (C-E) Immunofluorescence staining using antisera targeting (C) Nanog, Tra160; (D) Sox2, SSEA4; (E) Oct4 alkaline phosphatase confirms pluripotency. Nuclei are counterstained with Hoechst 33342. (F-G) Undirected EBs were cryosectioned and immunostained for the 3 developmental germ layers: endoderm (Sox17), mesoderm (SMA), and ectoderm (Tuj1). (H-J) Teratomas were sectioned and hematoxylin and eosin stained, and show evidence of the presence of the three developmental germ layers as indicated. (K-L) Nanostring analysis for endogenous stem cell genes (K), and shutoff of Sendai transgenes (L). (M) Immunofluorescence staining using antisera targeting neuron-specific class III β-tubulin (Tuj1, green) and the neural progenitor marker paired box 6 (PAX6) demonstrates directed neuronal differentiation (21 days). Nuclei are counterstained with DAPI. (N) This iPSC line displays a normal female karyotype.

FIGS. 3A-3U. Characterization of additional dura-derived iPSCs. All scale bars are 100 μm and representative clones are shown. ASC19D-ALS (clone 1) is from a sporadic ALS case. ASC27D-HD (clone 2) is from a Huntington's disease case. ASC30D-PD (clone 1) is from a sporadic Parkinson's disease case. (A-C) Outgrowth with fibroblast morphology from dura mater. (D-L) Immunostaining for pluripotency markers as indicated. AP stands for alkaline-phosphatase. Undirected EBs were cryosectioned and immunostained for the 3 developmental germ layers: endoderm (AFP), mesoderm (SMA), and ectoderm (Tuj1). (S-U) iPSC-derived neurons (Tuj1) and neural progenitors (Pax6) after 14 days of directed neuronal differentiation.

FIGS. 4A-4C. Characterization of scalp and dura-derived iPSCs. This figure shows the relative expression of fibroblast genes for scalp and dural outgrowths from the same MSA patient, as shown in FIG. 1, as well as karyotype data for these lines. (A) RNA isolated from scalp and dural fibroblast lines from from the same patient (ASC2S and ASC2D) were analyzed on the Illumina HumanHT-12-14 BeadChip platform as biological triplicate samples. Upper panel: Scatter plot (log₂ scale) comparing gene expression in scalp and dural fibroblasts reveals an intermediate degree of correlation (r²=0.86). The upper and lower lines mark the 3-fold expression difference boundaries. Lower panel: Scorecard analysis comparing scalp and dural fibroblast microarray expression data for representative fibroblast markers. (B-C) ASC2D-MSA-CP (clone 2) and ASC2S-MSA-CP (clone 3) both display a normal female karyotype.

FIGS. 5A-5B. Additional characterization of dura-derived iPSCs. This figure shows additional Nanostring analysis for endogenous stem cell genes and shutoff off Sendai transgenes for additional iPSC lines. (A) Nanostring analysis for endogenous stem cell genes and (B) shutoff of Sendai transgenes for ASC19D-ALS (clone 1), ASC27D-HD (clone 2), and ASC30D-PD (clone 1). Hues16 and Hues42 were used as positive controls for endogenous stem cell genes, unrelated fibroblasts as a negative control, and infected unrelated fibroblasts as a positive control for Sendai transgene expression.

FIGS. 6A-6B. Outgrowths from additional tissues. Both pictures are from 17 days post-plating under similar conditions to dural outgrowths, as described in Example 1. (A) Cells grew out of a non-cryprotected large cerebral artery from an ALS subject. (B) Cells grew out of non-cryoprotected choroid plexus from a frontotemporal lobar degeneration/motor neuron disease subject.

### DETAILED DESCRIPTION

Many of the embodiments of the present invention are described in the above Summary of the Invention section of this application, as well as in the Examples, Drawings, and Claims. This Detailed Description section provides additional description relating to various aspects of the invention, and is intended to be read in conjunction with the other sections of the present application.

The present invention provides cells, including but not limited to, pluripotent stem cells and differentiated cells, derived from tissue samples that had been frozen without a cryoprotective agent, cell panels comprising such cells, model systems comprising such cells, and methods for making and using such cells, cell panels and model systems, for example for in in vitro screening methods.

### Cells

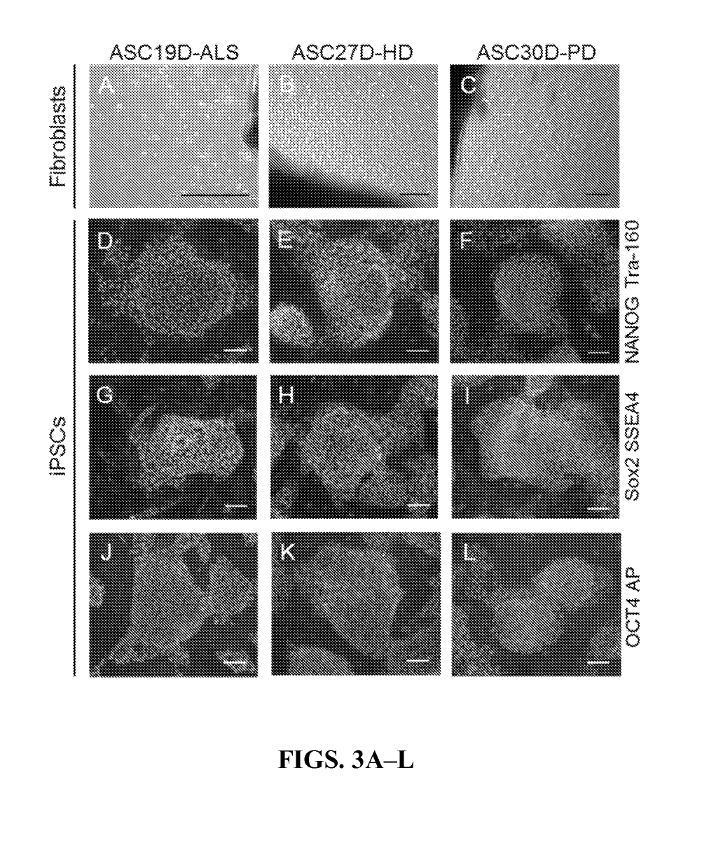

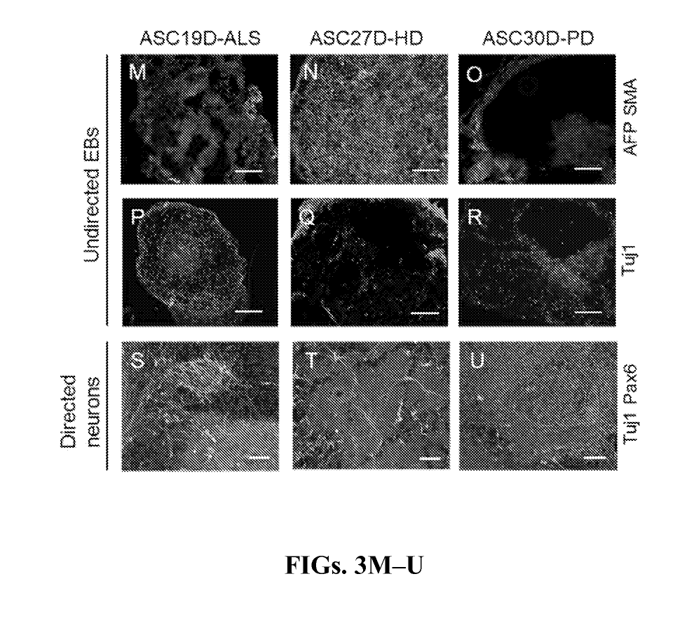

The present invention provides cells, and populations of cells (such as cell lines), derived from a tissue sample that had been frozen without a cryoprotective agent. In some embodiments, the cells are cultured as outgrowths directly from the tissue sample, for example, fibroblasts or cells with fibroblast-like morphology (see, for example, FIGS. 3A-3C (dural fibroblasts) and 6A-6B (outgrowths of large cerebral artery and leptomeninges). In some embodiments, the cells are pluripotent stem cells, such as induced pluripotent stem cells (iPSCs). In such embodiments, the pluripotent stem cells may be generated from cells derived directly from the tissue, such as from fibroblast outgrowths. In some embodiments, the cells are differentiated cells, such as neurons. In some embodiments, the differentiated cells are derived from pluripotent stem cells, such as induced pluripotent stem cells, derived from the tissue sample. In some embodiments, the differentiated cells are derived by trans-differentiation of differentiated somatic cells grown from the tissue sample, or by trans-differentiation of pluripotent cells (such as pluripotent stem cells or induced pluripotent stem cells) derived from the tissue sample, for example induced pluripotent stem cells generated from somatic cells grown from the tissue sample. In some embodiments, the cells comprise one or more exogenously-introduced reporter molecules, exogenously-introduced reprogramming factors, or exogenously-introduced differentiation factors.

A pluripotent stem cell is a cell that can a) self-renew and b) differentiate to produce cells of all three germ layers (i.e. ectoderm, mesoderm, and endoderm). The term “induced pluripotent stem cell” encompasses pluripotent stem cells, that, like embryonic stem cells (ESC), can be cultured over a long period of time while maintaining the ability to differentiate into cells of all three germ layers, but that, unlike ES cells (which are derived from the inner cell mass of blastocysts), are derived from somatic cells, that is, cells that had a narrower, more defined potential and that in the absence of experimental manipulation could not give rise to cells of all three germ layers. iPSCs generally have an hESC-like morphology, growing as flat colonies with large nucleo-cytoplasmic ratios, defined borders and prominent nuclei. In addition, iPSCs generally express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, iPSCs, like other pluripotent stem cells, are generally capable of forming teratomas. In addition, they are generally capable of forming or contributing to ectoderm, mesoderm, or endoderm tissues in a living organism.

Illustrative iPSCs are cells into which the genes Oct-4, Sox-2, c-Myc, and Klf have been transduced, as described by Takahashi and Yamanaka (Cell 126(4):663-76 (2006), the contents of which are hereby incorporated by reference in their entirety). Other exemplary iPSCs are cells into which OCT4, SOX2, NANOG, and LIN28 have been transduced (Yu, et al., Science 318:1917-1920 (2007), the contents of which are hereby incorporated by reference in their entirety). One of skill in the art would know that various different cocktails of reprogramming factors can be used to produce iPSCs, such as factors selected from the group consisting of OCT4, SOX2, KLF4, MYC, Nanog, and Lin28. The methods described herein for producing iPSCs are illustrative only and are not intended to be limiting. Rather any suitable methods or cocktails of reprogramming factors known in the art can be used. In embodiments where reprogramming factors are used, such factors can be delivered using any suitable means known in the art. For example, in some embodiments any suitable vector, such as a Sendai virus vector, may be used. In some embodiments reprogramming factors may be delivered using modified RNA methods and systems. A variety of different methods and systems are known in the art for delivery of reprogramming factors and any such method or system can be used.

Any culture medium suitable for culture of cells, such as pluripotent stem cells, may be used in accordance with the present invention, and several such media are known in the art. For example, a culture medium for culture of pluripotent stem cells may comprise Knockout DMEM, 20% Knockout Serum Replacement, nonessential amino acids, 2.5% FBS, Glutamax, beta-mercaptoethanol, 10 ng/microliter bFGF, and antibiotic. The employed medium may also be a variation of this medium, for example without the 2.5% FBS, or with a higher or lower % of knockout serum replacement, or without antibiotic. The employed medium may also be any other suitable medium that supports the growth of human pluripotent stem cells in undifferentiated conditions, such as mTeSR (available from STEMCELL Technologies), or Nutristem (available from Stemgent), or ES medium, or any other suitable medium known in the art. Other exemplary methods for generating/obtaining pluripotent stem cells from a population of cells grown out of a tissue sample that had been frozen with or without a cryoprotective agent, are provided in the Example of the present application.

In some embodiments, pluripotent stem cells are differentiated into a desired cell type, for example, neurons, oligodendrocytes, cardiomyocytes, or any other desired cell type. Differentiated cells provided by the invention can be derived by various methods known in the art using for example, adult stem cells, embryonic stem cells (ESCs), epiblast stem cells (EpiSCs), and/or induced pluripotent stem cells (iPSCs; somatic cells that have been reprogrammed to a pluripotent state). Methods are known in the art for directed differentiation or spontaneous differentiation of pluripotent stem cells, for example by use of various differentiation factors. Differentiation of pluripotent stem cells may be monitored by a variety of methods known in the art. Changes in a parameter between a stem cell and a differentiation factor-treated cell may indicate that the treated cell has differentiated. Microscopy may be used to directly monitor morphology of the cells during differentiation.

Cells provided by the invention can be used for any suitable purpose as known in the art or within the scope of the present invention as further described herein.

### Cell Panels

The present invention provides panels of cells that comprise at least two populations of cells derived from tissue samples that had been frozen without a cryoprotective agent. In some embodiments, a cell panel comprises cells outgrown directly from the tissue sample, such as fibroblasts. In some embodiments, a cell panel comprises pluripotent stem cells, such as induced pluripotent stem cells. In some embodiments, a cell panel comprises differentiated cells, such as neurons or any other desired cell type.

In some embodiments, a cell panel comprises at least two populations of cells, at least 10 populations of cells, at least 50 populations of cells, at least 100 populations of cells, at least 500 populations of cells, at least 1000 populations of cells, or at least 2000 populations of cells. In some embodiments, a cell panel comprises 6, 12, 24, 48, 96, 384, or 1536 populations of cells. In some embodiments, a cell panel comprises populations of cells in duplicate or triplicate or the like. In some embodiments, each population of cells in a cell panel is derived from a different tissue sample, or each population of cells in a cell panel is derived from the same tissue sample, or the cell panel comprises populations of cells derived from at least two tissue samples. In some embodiments, the cell panel comprises populations of cells derived from one or more tissue samples obtained from the same subject. In some embodiments, the cell panel comprises populations of cells derived from one or more tissue samples obtained from multiple different subjects, for example at least two different subjects. In some embodiments, cells in each cell population comprise one or more exogenously-introduced reporter molecules or exogenously-introduced reprogramming factors.

Cell panels can be assembled or organized or prepared in any suitable platform or format, a variety of which are known in the art. In some embodiments, cell panels are situated on a solid subtrate or in vessel such as a multi-well plate (e.g., 6-well, 12-well, 24-well, 48-well, 96-well, 384-well, or 1536-well), slide (e.g., single chamber or multi-chamber), or chip. In such embodiments, each cell population is preferably situated in its own well or chamber or otherwise separated from other populations in the panel so as to prevent or minimize mixing of populations. In some embodiments, each cell population is contained in a separate vessel. In some embodiments, cell panels comprise live cells or frozen cells or embedded cells (e.g., paraffin-ebedded) or any combination thereof. In some embodiments, the cell panels are suitable for high-throughput screening. In some embodiments, cell panels are suitable for detection and/or analysis of expression of DNA, RNA, protein or the like, such as by microarray analysis, immunohistochemistry, in situ hyridization and other methods known in the art. In some embodiments, cell panels comprise cells derived from tissue samples obtained from subjects that have or had a disease or disorder (e.g., experimental samples) and/or cells derived from tissue samples obtained from subjects that do not have or did not have the disease or disorder (e.g., control samples).

Cell panels provided by the invention can be used for any suitable purpose in addition to those described above, including but not limited to drug screening, disease diagnosis/prognosis, pathology, diagnosis confirmation, and genotype and/or phenotype analyses.

### Model Systems

The present invention provides model systems comprising (i) one or more populations of cells, or one or more cell panels, wherein the the cells were derived from a tissue sample that had been frozen without a cryoprotective agent; (ii) information obtained from and/or associated with the cells and/or the tissue sample.

The model systems of the invention can comprise any cells, populations of cells, or cell panels encompassed by the invention, including the cells, populations of cells, or cell panels described herein. In some embodiments, a model system comprises cells outgrown from the tissue sample (for example, fibroblasts), pluripotent stem cells (such as induced pluripotent stem cells), or differentiated cells. In some embodiments, the model system comprises an experimental component (or test component) and a control component. In some such embodiments, the experimental component (or test component) may comprise cells or populations of cells derived from a tissue sample that was frozen without a cryoprotective agent and where the tissue samples are from subjects that have or had a disease or disorder; and the control component may comprise cells or populations of cells derived from a tissue sample that was frozen without a cryoprotective agent and where the tissue samples are from subjects that do not have or did not have the disease or disorder.

In an exemplary model system for Alzheimer's disease, the experimental component may comprise a panel of neurons derived from tissue samples that were frozen without a cryoprotectant, where the tissue samples were orginally obtained from different subjects with Alzheimer's disease confirmed by post-mortem tissue analysis, and associated information relating to the tissue samples; and the control component may comprise neurons derived from tissue samples that were frozen without a cryoprotectant where the tissue samples were orginally obtained from different subjects that do not or did not have Alzheimer's disease, and associated information relating to the tissue samples. Such a model system can comprise live neurons that can be assessed or studied under desired conditions, including genetic manipulation, exposure to toxic agents or therapeutic agents (or potentially toxic or therapeutic agents) such that one can determine or measure the effect of such conditions on experimental cells and control cells, for example by measuring any suitable property of such cells, including, but not limited to, electophysiological properties, or properties relating to neurodegeneration, neuronal survival, gene expression, protein expression or the like. This example is illustrative only, and a person of skill in the art will appreciate that the present invention encompasses countless variations to such an exemplary model system.

In some embodiments, a model system further comprises a tissue sample from which a population or populations of cells in the panel was derived. Including a sample of the original tissue sample in the model system can enable comparison or confirmation of findings in cells derived from the tissue with the original tissue sample itself. One can look back in the tissue sample to verify findings in the model system, and findings can further be correlated or cross-validated with information associated with the tissue sample.

In some embodiments, model systems comprise a data panel comprising information associated with each cell population in the cell panel or each tissue sample or subject from which each cell population was derived. In some embodiments, the data panel comprises information about the subject's age, sex, race, genotype, phenotype, environment, medical history, health, family history, disease status, treatment history, histopathology, diagnosis, disease classification, disease stratification, disease grading, disease severity, or disease progression. In some embodiments, the data panel comprises information about the subject, the tissue sample, or the cell population that was obtained post-mortem. The information used in the model systems of the invention can comprise any information associated with (or relating to or describing) the tissue sample, where such information may have been obtained from the biobank from which the tissue sample was obtained. In some embodiments, the information is related to the subject from which the tissue sample was originally obtained, for example, the subject's physical traits (e.g., age, weight, and/or ethnicity), lifestyle and/or habits (e.g., smoker or non-smoker), disease diagnosis, and other information as appropriate and/or available. In some embodiments, the information is related to the tissue sample itself, for example, histology, pathology, genetic analysis, transcriptional analysis, proteomic analysis, and the like. In some embodiments, the information is related to a disease or disorder, for example, information related to the diagnosis, post-mortem diagnosis, classification, severity, stratification, or grading of a disease or disease, and the like. In some embodiments, the information is related to a cell or population of cells derived from the tissue sample, for example, methods and/or reagents for cell culture, reprogramming (e.g., for iPSCs), and/or differentiation (i.e., for differentiated cells), genetic analysis, transcription analysis, proteomic analysis, functional analysis, and the like.

The successful generation of living somatic cells from frozen non-cryoprotected tissue samples, and the subsequent successful derivation of iPSCs and differentiated cells from such somatic cells, provides a surprising new source of well-characterized samples that can be used to develop new and/or improved model systems for diseases and disorders, such as those that, prior to the present invention, were not accurately or definitively reproduced in a cell-based system, or that cannot be correctly diagnosed until post-mortem, or for rare diseases that have a low frequency in clinical populations.

### Tissue Samples

The present invention is based on the unexpected finding that live cells can be successfully generated from tissue samples that had been frozen without a cryoprotective agent. As used herein, “cryoprotective agent” and “cryoprotectant” are used interchangeably to refer to an agent that is added to a tissue sample in preparation for freezing in order to reduce or prevent the formation of ice in the tissue sample. A variety of cryoprotective agents are known in the art including synthetic cryoprotective agents and natural cryoprotectants. Non-limiting examples of cryoprotective agents include dextrans, dimethyl sulfoxide (DMSO), ethylene glycol, glycerol, hydroxyethyl starch, polyvinylpyrrolidone, propylene glycol and sucrose. Ice formation in a tissue sample during freezing is a critical factor that is known to severely restrict the viability (e.g., the ability of the preserved tissue or cells to perform their normal function upon return to physiological conditions) of the tissue sample, or the viability of cells within the tissue sample, after freezing and thawing. (See, for example, Brockbank & Taylor, “Tissue Preservation.” Advances in Biopreservation (2006):157-196, the contents of which are hereby incorporated by reference in their entirety.)

Tissue samples frozen without a cryoprotective agent are highly susceptible to damage or injury by freezing-induced chemical and mechanical stresses, such as the formation and aggregation of ice, cellular dehydration, intracellular crystallization, and thermal shock. The intent of cryopreservation (e.g., freezing of a tissue sample in the presence of one or more cryoprotective agents), as opposed to mere freezing (e.g., without a cryoprotective agent) is to maintain a biological tissue sample in a living state at cryogenic temperatures by protecting the tissue sample from freezing-induced stresses and damage, such that the tissue will retain viability when returned to physiological conditions. (See, Bakhach J., Organogenesis, 2009, 5(3):119-126.) Thus tissue samples frozen without the addition of a cryoprotective agent would not be expected to retain viability when returned to physiological conditions or similar conditions when cultured in vitro.

Tissue samples that are frozen or had previously been frozen can be obtained from any suitable source. Preferably, a tissue sample is obtained from a biobank such that the sample is obtained together with associated information and/or data on record at the biobank. As used herein, “biobank,” “tissue bank,” and “biorepository” are used interchangably to refer to an organized collection of frozen biological samples together with associated information and/or data on record for such samples.

Tissue samples contemplated by the present invention can comprise any type of biological tissue including epithelial tissue, connective tissue, nervous tissue, or muscle tissue. In some embodiments, the tissue sample comprises skin, vascular tissue, epithelial lining of a cavity or organ, brain tissue, liver tissue, eye tissue, lung tissue, pancreatic tissue, muscle tissue, or heart tissue. In some embodiments, the tissue sample comprises tissue obtained from a neoplasm (such as a biopsy sample), for example, a tumor, cyst, lump or the like. In such embodiments, the neoplasm can be benign, potentially malignant/pre-cancerous, or malignant/cancer. In some embodiments, the tissue sample comprises dura mater. As used herein, “tissue sample” includes samples of solid tissues and also includes samples of biological fluids (for example, blood (e.g., umbilical cord blood), urine, ascites, saliva, and semen) and biological samples derived from a tissue sample (for example, blood derivatives, purified or partially-purified cells, DNA, RNA, or protein).

Tissue samples used in accordance with the invention may have been frozen at any suitable temperature. For example, a tissue sample may have been frozen by methods using liquid nitrogen (for example, a liquid nitrogen vapor sandwich method) which has a temperature of −196 degrees Celsius. In some embodiments, a tissue sample has or had been frozen at a temperature below zero degrees Celsius, below −20 degrees Celsius, below −40 degrees Celsius, below −60 degrees Celsius, below −80 degrees Celsius, below −100 degrees Celsius, below −120 degrees Celsius, below −140 degrees Celsius, below −160 degrees Celsius, below −180 degrees Celsius, or below −200 degrees Celsius. In some embodiments, the tissue sample has or had been frozen at about −80 degrees Celsius. In some embodiments, the tissue sample has or had been frozen at about −196 degrees Celsius. In some embodiments, the tissue sample has or had been frozen for more than 1 month, for more than 6 months, for more than 1 year, more than 5 years, more than 10 years, or more than 20 years. In some embodiments, the tissue sample has or had been frozen for less than 1 month or less than 1 week.

A tissue sample will preferably be a sample of human tissue originally obtained from a human subject, however a tissue sample can be from any suitable subject. The term “subject” as used herein encompasses mammals, including but not limited to, humans, non-human primates, and laboratory animals, including rodents such as mice and rats. In some embodiments, the subject was deceased at the time the sample was obtained (e.g. a post-mortem sample). A tissue sample can originate or be obtained from any part of a subject's body, including but not limited to skin, brain, lungs, liver, pancreas, heart, muscle, breast, bladder, colon, male or female reproductive tracts (including, but not limited to, prostate, testes, cervix, ovaries, uterus), esophagus, eyes, or blood vessels. In some embodiments, the subject has or had a disease or disorder, or the subject is or was healthy or does not have or did not have a given disease or disorder.

### Diseases

The cells, cell panels, model systems and methods provided by the present invention can be used within the context of a wide variety of diseases or disorders, including but not limited to neurological diseases or disorders, diabetes, cancer, immune diseases or disorders, vascular diseases or disorders, heart diseases or disorders, or HIV/AIDS. In some embodiments, the disease or disorder is a degenerative disease or disorder, for example, comprising degeneration of nervous tissue or muscle tissue. In some embodiments, the disease or disorder comprises a neurological disease or disorder, including but not limited to Alzheimer's disease, Parkinson's disease, amyotrophic lateral sclerosis (ALS), Huntington's disease, multiple systems atrophy (MSA), diffuse Lewy body disease (DLBD), vascular dementia, or frontotemporal lobar degeneration. In some embodiments, the disease or disorder is cancer, including but not limited to breast cancer, leukemia, lymphoma, cervical cancer, prostate cancer, colon cancer, bladder cancer, endometrial cancer, gastric (stomach) cancer, glioma, esophageal cancer, skin cancer, lung cancer, pancreatic cancer, or skin cancer (melanoma or non-melanoma).

Generation of Cells from Frozen Tissue

In some embodiments the present invention provides methods for generating a population of cells from a tissue sample that had previously been frozen. Any suitable methods known in the art for obtaining populations of cells from frozen tissue samples can be used. For example, standard methods known in the art for thawing tissue samples and manipulating tissue samples so as to obtain and culture cells therefrom can be used in conjunction with the present invention. Some suitable methods are described in the Example section of this application. However, other suitable methods will be known to those in the art.

In some embodiments the present invention provides methods for generating a population of cells, such a pluripotent cells or differentiated cells, from a sample of dura mater that had been frozen. In some embodiments, the dura mater sample was frozen without a cryoprotective agent. In some embodiments, the dura mater sample was frozen with a cryoprotective agent. A variety of cryoprotective agents are known in the art, for example, dextrans, dimethyl sulfoxide (DMSO), ethylene glycol, glycerol, hydroxyethyl starch, polyvinylpyrrolidone, propylene glycol and sucrose.

In some embodiments, the method for generating a population of pluripotent stem cells from frozen dura mater, comprises (a) obtaining a dura mater sample, wherein the dura mater sample is frozen, or had previously been frozen; (b) culturing cells from the dura mater sample in vitro so as to obtain a population of cells from the dura mater sample; and (c) deriving a population of pluripotent stem cells from the population of cells of step (b). In some embodiments, the population of cells of step (b) comprises cells outgrown directly from the dura mater sample (for example, dural fibroblasts, as described in Example 1). In some embodiments, the deriving of step (c) comprises reprogramming the cells cultured from the dura mater sample in step (b) so as to generate induced pluripotent stem cells. Exemplary methods for generating iPSCs by reprogramming are illustratively described herein; and these and other methods are well known in the art. (For a review, see, Rao & Malik, 2012, Assessing iPSC reprogramming method for their suitability in translational medicine, J Cell Biochem 113(10):3061-3068, the contents of which are hereby incorporated by reference in their entirety.)

### Methods

The present invention provides method for making and using cells, cell panels, and model systems of the invention.

In one embodiment, the invention provides a method for generating a population of cells from a tissue sample that was frozen without a cryoprotectant, comprising culturing cells from the tissue sample so as to obtain a population of cells from the tissue sample. In some embodiments, the cells are somatic cells (such as fibroblasts) that are cultured as outgrowths from the tissue sample. In another embodiment, the invention provides a method for generating a population of pluripotent stem cells from a tissue sample that was frozen without a cryoprotectant, comprising culturing cells from the tissue sample so as to obtain a population of cells from the tissue sample, and then deriving a population of pluripotent stem cells from that population of cells. In some embodiments, the pluripotent stem cells are induced pluripotent stem cells. In such embodiments, the method further comprises a step of reprogramming the cells cultured from the tissue sample so as to generate induced pluripotent stem cells.

In one embodiment, the invention provides an in vitro screening method, comprising (a) contacting one or more populations of pluripotent cells, multipotent cells (such as in an embryoid body) or differentiated cells with a candidate agent (such as a candidate therapeutic agent), wherein each population of differentiated cells was derived from a population of pluripotent stem cells, wherein each population of pluripotent stem cells was derived from a tissue sample, wherein the tissue sample had been frozen without a cryoprotective agent, and wherein the tissue sample is from a subject that has or had a disease or disorder; and (b) observing or determining whether the candidate agent has an effect in one or more assays. In some embodiments, the pluripotent stem cells are induced pluripotent stem cells. The step of observing or determining whether candidate agent has an effect can be performed using any suitable assay known in the art to evaluate any desired property of the cells. In some embodiments such assays may involve evaluating one or more reporter molecules which may have been exogenously introduced into the cells, such as one or more reporter genes, such as fluorescently labelled reporter genes, or any other reporter molecule that is desired. In some embodiments such reporter molecules encompass, or are labelled with, a detectable moiety such as a fluorescent moiety or any other suitable moiety.

In some embodiments, the differentiated cells are neurons. In such embodiments, the assay may comprise monitoring one or more properties of neuronal cells or one or more indicators of neurodegeneration or neuronal survival. In some embodiments, the tissue sample comprises dura mater. In some embodiments, the disease or disorder is a neurological disease, including but not limited to Alzheimer's disease, Parkinson's disease, amyotrophic lateral sclerosis (ALS), Huntington's disease, multiple systems atrophy (MSA), diffuse Lewy body disease (DLBD), vascular dementia, or frontotemporal lobar degeneration. In some embodiments, the disease or disorder is cancer, including but not limited to breast cancer, leukemia, lymphoma, cervical cancer, prostate cancer, colon cancer, bladder cancer, endometrial cancer, gastric (stomach) cancer, glioma, esophageal cancer, skin cancer, lung cancer, pancreatic cancer, or skin cancer (melanoma or non-melanoma). In some embodiments, the disease or disorder is diabetes.

For any given cell type, one of skill in the art will recognize assays and other methods that can be used within the context of the invention to determine the effect of a given candidate agent.

### EXAMPLE

The invention is further described in the following non-limiting Example.

### Example

### Generation of Induced Pluripotent Stem Cell Lines from Non-Cryoprotected Biobanked Dura Mater

The findings presented in this Example are further described in Sproul et al.: Generation of iPSC lines from archived non-cryoprotected biobanked dura mater. Acta Neuropathologica Communications 2014 2:4, the contents of which are hereby incorporated by reference in their entirety.

Human iPSC-derived neural cells are attractive models for Alzheimer's disease and other neurodegenerative diseases because they can be used for cellular investigation of mechanisms and drug screening in vitro. While, to date, most human iPSC models have been derived from rare monogenetic familial forms of neurodegenerative disease, most patients have sporadic disease forms for which post-mortem neuropathological examination is essential for definitive diagnosis. In some cases, tissue from patients with similar symptoms may exhibit quite different pathology. For instance, vascular dementia or frontotemporal lobar degeneration, which can clinically present as AD, may not be correctly diagnosed until post mortem examination of the brain. If tissue from deceased patients that had undergone neuropathological evaluation could be used to generate iPSCs, knowledge of the definitive diagnosis as well as potential stratification of sporadic patients could guide the selection and subsequent use of the cell lines to be made. While generation of iPSCs from fresh autopsy tissue has recently been reported [1, 2], brain bank networks, which contain tens of thousands of samples, could provide a much larger, and more immediate, source of tissue. As these biobanked tissues have not been specifically processed for the derivation of living cells, it was investigated whether it is possible to use them to isolate somatic cells and subsequently reprogram these into iPSCs. This would allow access to large numbers of neuropathologically defined cases, including patients with rare diseases whose frequency is low in clinical populations.

### Methods

Cell Culture. All media components are commercially available and were obtained from Life Technologies unless otherwise indicated. Dural/scalp outgrowths, established fibroblast lines, and mouse embryonic fibroblasts (MEFs, GlobalStem) were grown in fibroblast media, defined as the following: DMEM/10% FBS/Glutamax (2 mM)/2-Mercaptoethanol (0.1 mM)/Penicillin-streptomycin (100 U/mL-0.1 mg/mL). For initial plating of new dural and scalp samples, the tissue was first grown in biopsy media: DMEM/10% FBS/Glutamax (2 mM)/2-Mercaptoethanol (0.1 mM)/MEM non-essential amino acids (0.1 mM)/antibiotic-antimycotic (1×)/Nucleosides (1×, Millipore). Reprogrammed iPSCs were maintained on MEFs, in HUESM: KO-DMEM/20% KSR/Glutamax (2 mM)/2-Mercaptoethanol (0.1 mM)/bFGF (10 ng/ml)/Penicillin-streptomycin (100 U/mL-0.1 mg/mL). iPSCs were enzymatically passaged using TrypLE and replated in the presence of a Rho-kinase inhibitor (Y27632, Stemgent). Karyotyping and DNA fingerprinting of fibroblasts and iPSCs was performed by Cell Line Genetics (Madison, Wis.). Directed and undirected differentiations are described below.

Generation of postmortem tissue outgrowths. De-identified donated postmortem brain tissue was obtained. Neuropathological examination was per standardized protocols [3, 4]. For the pilot study, scalp and dural tissue from the same patient was frozen at the time of autopsy in the presence of 10% DMSO/45% FBS/45% fibroblast media. Subsequent experiments utilized standard banked material that was frozen via a liquid nitrogen vapor sandwich method [3, 4]. Dural tissue was stored as rolled tissue (approximately 1 cm by 5 cm in 2 ml cryovials that have been stored at −80 degrees for 1-11 years. Samples were either thawed entirely for 1 min at 37° C. for processing or quickly removed from the vial with forceps while still frozen and a small piece was cut off using a scalpel, to preserve unthawed tissue for future use. Samples were washed twice with PBS and DMEM, then cut into smaller pieces, approximately 2-3 mm by 2-3 mm. One drop of sterile silicon grease was placed in the center of each well of a 6-well cell culture plate and four or five pieces of the tissue were placed around each drop. A coverslip was placed on top of the silicon/samples and 2 ml of biopsy culture media were added to each well. Samples were left undisturbed for five days and then checked for fibroblast outgrowth. Media was switched to fibroblast culture medium and changed every other day. Outgrowth was monitored and fibroblasts were passaged with TrypLE to a new 6-well culture dish when the coverslip and/or plate became at least 50% confluent. Fibroblast cultures were expanded and passaged until sufficient numbers were generated for reprogramming and cryogenic preservation.

Reprogramming using Sendai virus. Fibroblasts between passages 3 and 5 were plated into a 12-well cell culture plate format at 50,000 cells per well in fibroblast culture medium. CytoTune-iPS kits (Life Technologies) containing four Sendai virus vectors (Oct3/4, Sox2, Klf4, c-Myc) were used to infect fibroblasts at an MOI=3 (transduction volume based on the specific titer of each lot), in fibroblast media. The day after infection, an additional 1 mL of fibroblast media was added to the culture. The next day, the media was switched to HUESM and depending on the severity of Sendai toxicity, MEFs were overlaid on some of the cultures. The medium was changed every day until colonies appeared. Colonies were manually picked and expanded on MEFs.

Dural and scalp fibroblast gene expression profile. RNA was prepared using the RNeasy mini kit (Qiagen) per the manufacturer's instructions. cRNA was amplified using the Illumina TotalPrep RNA Amplification Kit (Ambion) and run on an Illumina HT_12_v4 BeadChip Array (Ilumina), as per the manufacturer's instructions. Analysis of microarray data was performed using Genome Studio software (Illumina).

Immunostaining for pluripotency markers and alkaline phosphatase assay. For pluripotency staining, cultures were fixed using 4% paraformaldehyde (PFA, Santa Cruz) for 12 min at room temperature. After multiple PBS washes the cells were treated with PBS containing 0.1% Triton X-100 (Sigma) and 10% normal donkey serum (Jackson Immuno Research) for 1 hr. Cells were then treated with primary antibodies including Tra-160 (1:200, Millipore), SSEA-4 (1:500, Abcam), Tra-181 (1:200, Millipore), OCT-4 (1:500, Stemgent), Nanog (1:100, Cell Signaling), and SOX-2 (1:500, Stemgent). Alexa-conjugated anti-mouse or anti-rabbit IgG secondary antibodies were used (Invitrogen). AP staining was performed with the Vector Red Alkaline Phosphatase Substrate Kit (Vector Laboratories) per the manufacturer's instructions. Nuclei were counterstained with Hoechst 33342 (Sigma). Primary antibodies Tra-160 and Tra-181 bind to different epitopes on the same pluripotency marker protein. Positive immunostaining with Tra-160 antibodies is shown and described herein. Positive immunostaining with Tra-181 antibodies was also observed in scalp and dural samples (see U.S. provisional patent application 61/834,745 filed on Jun. 13, 2013, the contents of which are hereby incorporated by reference in their entirety, and to which the present application claims priority).

NanoString nCounter assay. Total RNA was isolated from each iPSC line using the RNeasy kit (Qiagen) as per the manufacturer's instructions. 100 ng of RNA for each sample was analyzed with the NanoString nCounter system (NanoString, Seattle, Wash.) using a pre-designed codeset. The codeset contains 25 probes for detection of retroviral and Sendai viral transgenes, pluripotency, spontaneous differentiation markers, and housekeeping genes [5]. Data was normalized to the geometric mean using nSolver Analysis Software v1.0 (NanoString) and compared with previous runs of a Sendai-positive control line, a fibroblast line (1043), and two human ESC lines (HUES42 and HUES16).

In vitro pluripotency assay. Undirected embryoid bodies (EB) were formed by placing 10,000 iPSCs in multiple wells of a 96-well non-tissue culture treated V-bottom plate (Evergreen) containing HUESM plus 10 μM ROCKi (Stemgent), and underwent brief centrifugation. After 14 d of culturing EBs were transferred into a 6 well low attachment plates (Corning) and cultured for an additional 16 days. Once harvested EBs were fixed in 4% paraformaldehyde for 20 min at room temperature and processed in 15% and 30% sucrose solutions for one day each at 4° C. EBs were then embedded in O.C.T. (Sakura Finetek) and cryosectioned. The sections were blocked in PBS containing 0.1% Triton X-100 and 10% donkey serum for 1 hr at room temperature, followed by an overnight incubation at 4° C. with antibodies identifying the 3 germ layers: SMA (1:500, DAKO), AFP (1:500, DAKO) TuJ1 (1:500, Covance), Sox17 (1:500, R&D Systems). Alexa-conjugated anti-mouse or anti-rabbit IgG secondary antibodies were used (Invitrogen) along with Hoechst 33342 counterstain. Sections were set with Vectashield Hard Set Mounting Media (Vector Laboratories). Sox17 antibodies and AFP antibodies recognize marker proteins present in endoderm. Positive immunostaining with AFP antibodies is described and shown herein. Positive immunostaining with Sox17 antibodies was also observed in scalp and dural samples (see U.S. provisional patent application 61/834,745 filed on Jun. 13, 2013, the contents of which are hereby incorporated by reference in their entirety, and to which the present application claims priority).

Teratoma assay. Two confluent wells of iPSC line ASC-7D-AD (p.25) were chemically disassociated using dispase (Gibco) and centrifuged for 4 minutes at 800 rpm. For each well, cells were resuspended as clumps in 100 μL of HUESM and added to 100 μl of Matrigel (BD Biosciences) on ice. A three-month-old NSG immune-compromised mouse (Jackson Laboratory) was anaesthetized with isofluorane and injected subcutaneously with a cell-suspension on each dorsal flank, and was sacrificed 75 d post injection. The teratoma was manually extracted, fixed in 4% PFA overnight and embedded in paraffin. Sections were stained with hematoxylin and eosin and histologically examined for developmental germ layers.

Directed neuronal differentiation. MEFs were manually removed and iPSCs were brought to single cells suspension using Accutase (Life Technologies) and plated into a 48-well plate coated with polyornithine (100 μg/mL, Sigma Aldrich)/laminin (3 μg/mL, Invitrogen) at 25,000 cells per well, in mTeSRl (Stem Cell Technologies) and 10 μM ROCKi. Cells were allowed to recover for 2 days before being switched to custom mTeSRl (missing five growth factors, Stem Cell Technologies) containing 10 μM SB421542 (Stemgent) and 250 nM LDN193189 (Stemgent). Media was changed completely every 2 days. At Day 11, media was gradually switched with half feeds for the first two changes to neuronal differentiation medium (Neurobasal/B27 without retinoic acid/Glutamax (2 mM)/Penicillin-streptomycin (100 U/mL-0.1 mg/mL).

At days 14 and 21, wells were washed with PBS and fixed using 4% PFA for 12 min at room temperature. After three PBS washes, cells were blocked with 0.1% Triton X-100 and 10% normal donkey serum for 1 hour. Cells were treated with primary antibodies including PAX6 (1:300, Covance), Tuj1 (1:500, Covance), and Sox1 (1:400, Stemgent). Alexa-conjugated anti-mouse or anti-rabbit IgG secondary antibodies were utilized (Invitrogen).

### Results

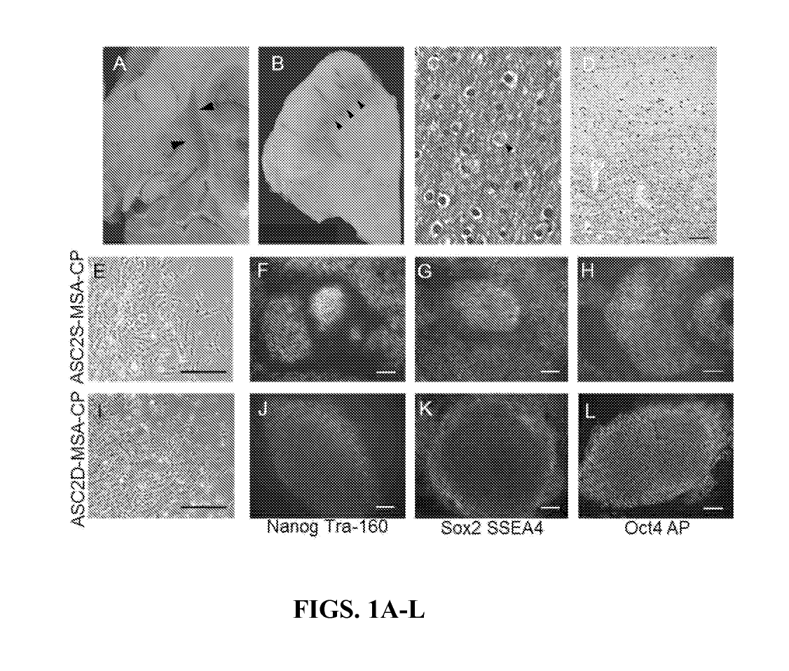

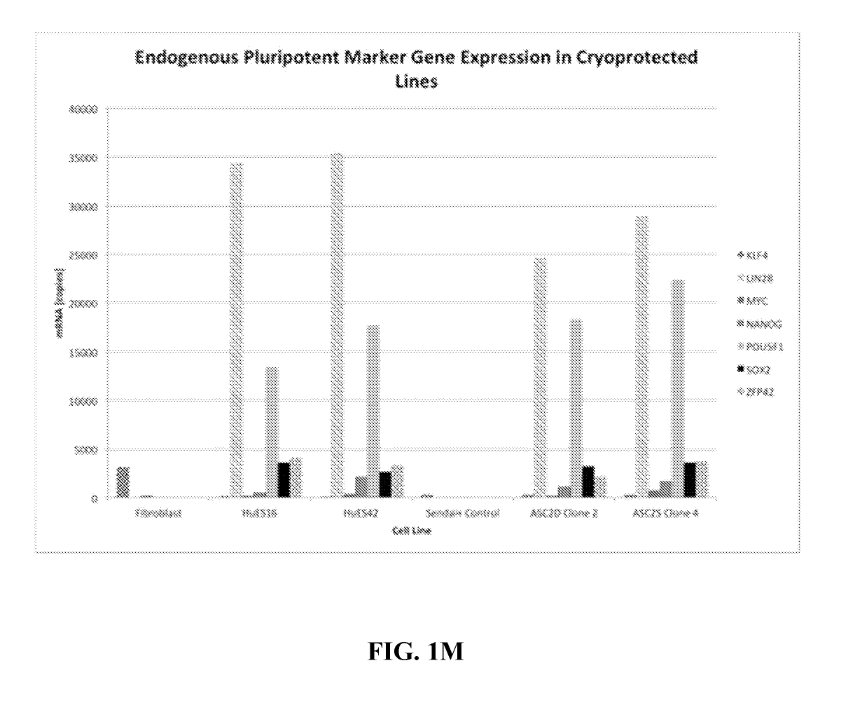

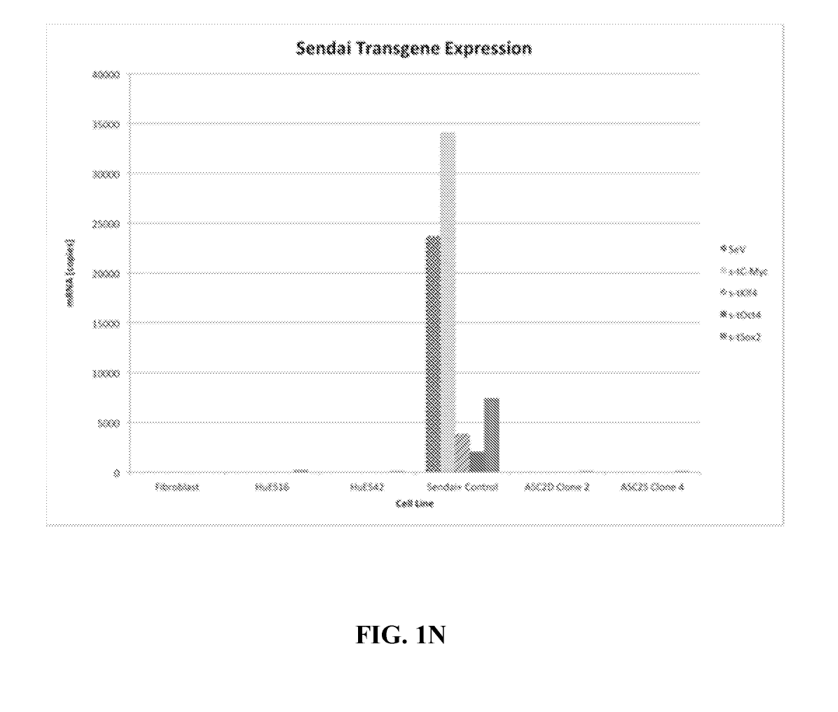

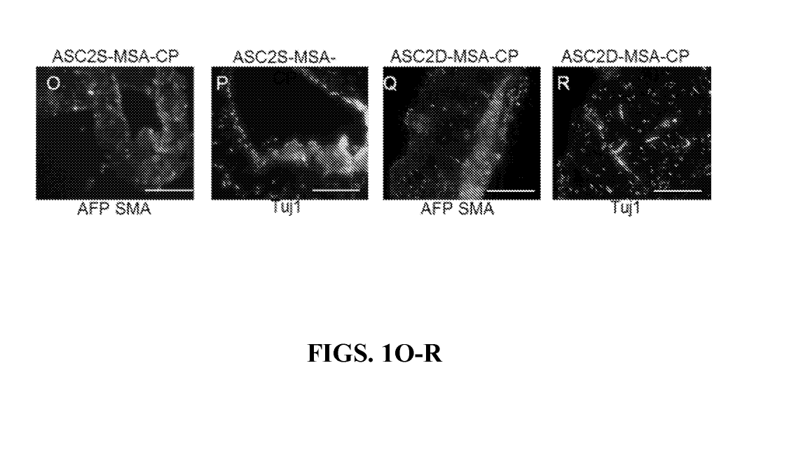

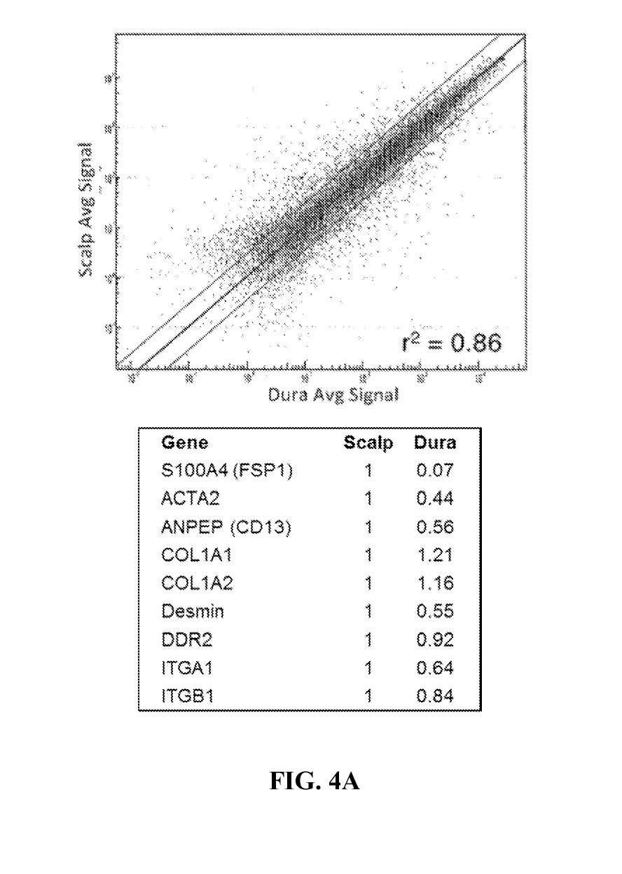

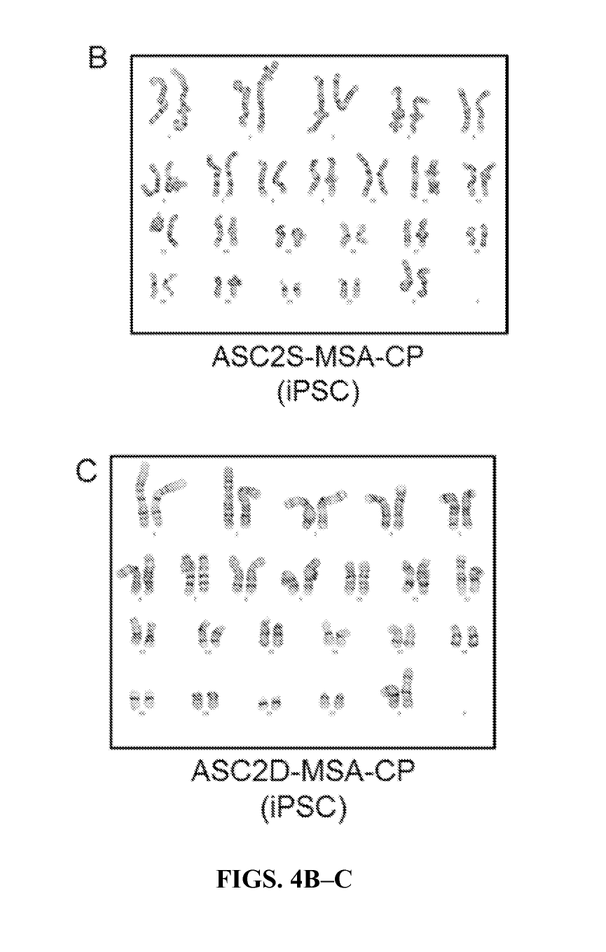

Generation of iPSCs from skin biopsies is routinely preformed in the art [5], but whether iPSCs with similar properties can be generated from meninges was unclear. To answer this question, the work described in this Example investigated the generation of dural fibroblasts from the skin (scalp) and cranial dura mater tissues from a random autopsy subject from a brain bank series. This patient had multiple system atrophy (MSA), a fatal disease characterized by glial cytoplasmic inclusions composed of α-synuclein that affects the striatum and other brain regions [6] (FIGS. 1A-1D). The tissue was frozen at autopsy using 10% DMSO/45% FBS/45% fibroblast media, a standard cryoprotection medium. Upon subsequent culture, samples taken from both tissues exhibited outgrowths with fibroblast morphology. Cell lines derived from these outgrowths were termed ASC2S-MSA and ASC2D-MSA (i.e. autopsy stem cell subject 2 skin and dura, ASC2S-MSA and ASC2D-MSA, respectively, FIGS. 1E and 1I). Gene expression profiling shows that the skin and dural fibroblast lines have similar but distinct gene expression profiles (correlation coefficient 0.86), indicating that the endosteal-derived dural fibroblasts have unique features. Comparing nine genes commonly used as functional fibroblast markers [7] reveals that most are present at similar levels in scalp and dural cells, with the exception of FSP1 (FIG. 4). In summary, both scalp and dural tissues yield outgrowths of fibroblast identity, albeit likely of different subclasses.

Next, studies were designed attempt to reprogram skin and dural lines into a pluripotent state using a Sendai virus integration-free method [8]. After viral infection, both ASC2S-MSA and ASC2D-MSA lines produced colonies with iPS-like morphology. Individual clones were manually picked, expanded, and characterized for stem cell properties such as pluripotency (FIGS. 1F-1H and 1J-1R). ASC2S-MSA-CP and ASC2D-MSA-CP iPSCs (cryoprotected) also displayed a normal female karyotype and fingerprinting confirmed that both lines were derived from the same subject (FIG. 4). These results show that both scalp and dural cells can be reprogrammed to produce high-quality iPSCs.

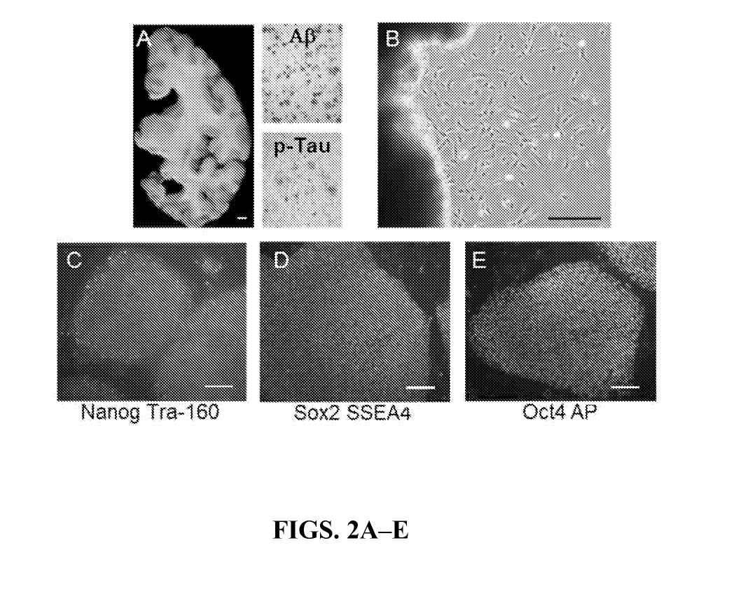

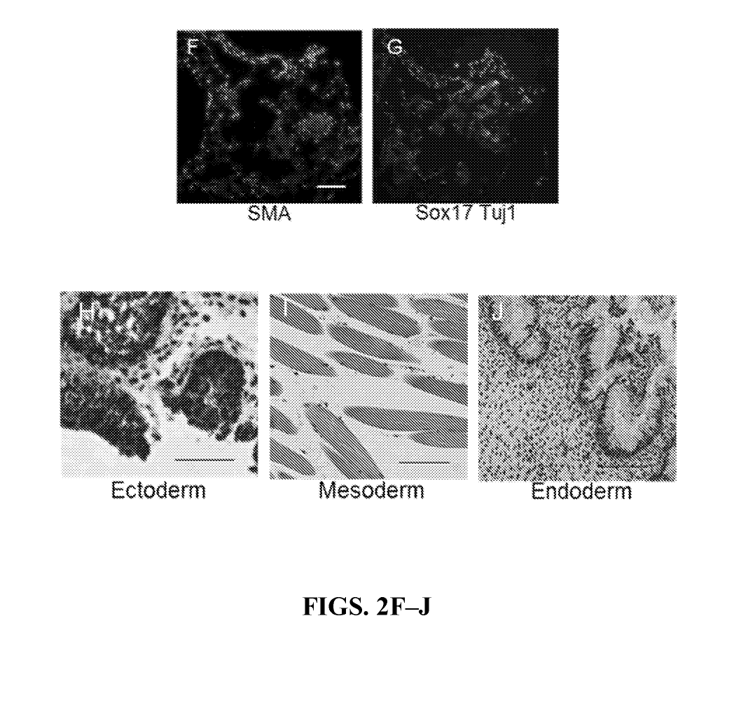

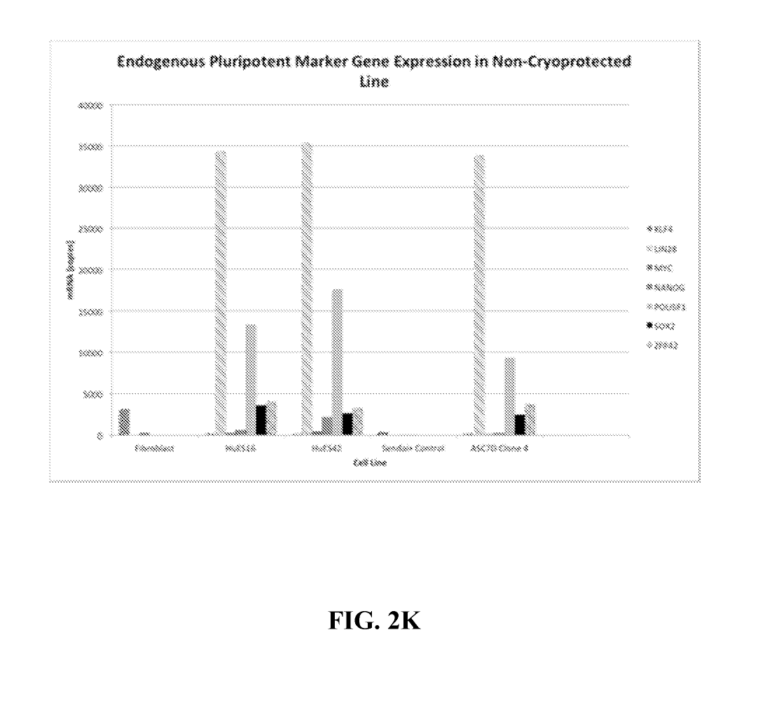

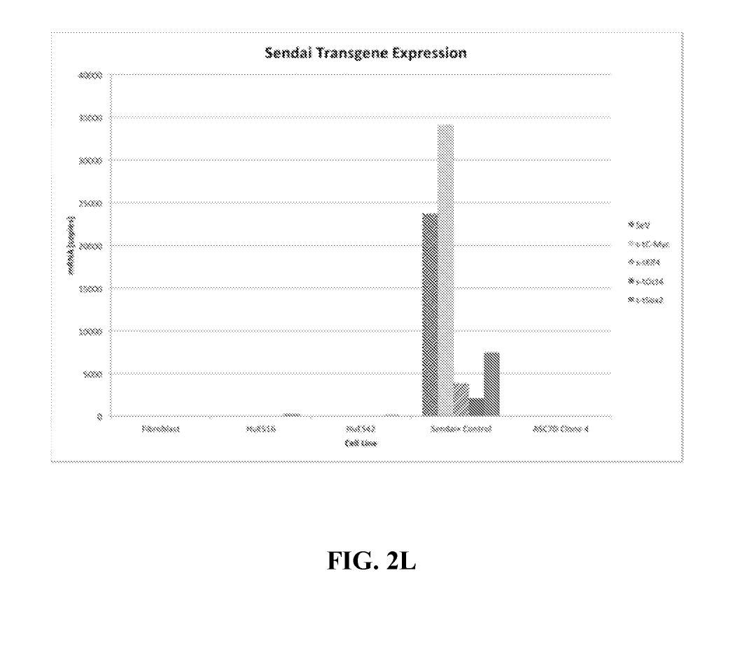

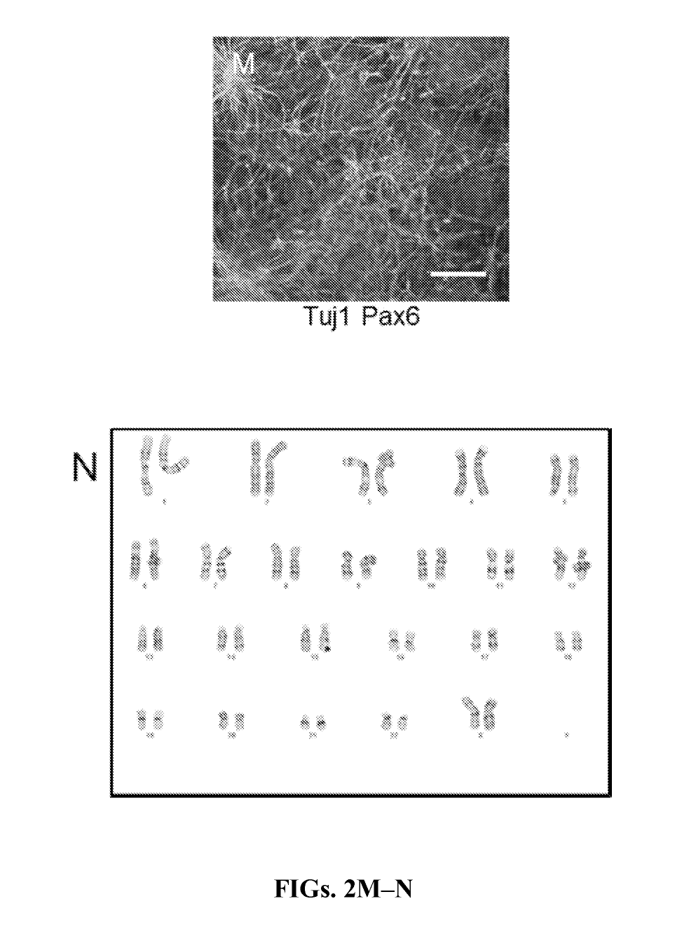

Based on these results, studies were designed to attempt to generate iPSCs from nine additional dural samples obtained from control individuals or from subjects with late-onset AD, all of which had been frozen and archived without a cryoprotectant such as DMSO, using a commonly utilized liquid nitrogen vapor sandwich method [3, 4]. Successful fibroblast outgrowths were obtained from four of the nine samples, although one of these outgrowths produced insufficient cells to reprogram (ASC9D). A second line was lost to contamination (ASC8D). When the remaining two lines were infected with Sendai virus, one (ASC7D-AD) formed colonies with iPSC morphology. This sample, stored at −80° C. for 9 years, was from a patient with pathological changes typical of AD (sporadic late-onset), including aggregated frequent amyloid plaques (CERAD plaque score C [9]) and severe accumulation and progression of phospho-tau inclusions (Braak neurofibrillary tangle stage VI, FIG. 2A [10]). Three individual clones were manually picked and expanded with the clone displaying the best morphology selected for further characterization, including confirmation of endogenous expression of stem cell genes by immunostaining and Nanostring analysis [5], verification of Sendai transgene shutoff, establishing pluripotency both in vitro (embryoid body assay) and in vivo (teratoma), and directed differentiation into neurons (FIGS. 2B-2M). ASC7D-AD had a normal female karyotype and matched the parent dural fibroblasts by fingerprinting (FIG. 2N).

To replicate the finding that non-cryoprotected archival dura mater can be used to generate iPSCs, as described in this Example, 18 additional biobanked cranial dura samples were acquired from a cohort of subjects with an assortment of neuropathologies. Fibroblast outgrowths were obtained from eight of these tissues (Table 1, below). As with the first attempt, it took longer to produce outgrowths than fresh skin biopsies: 11-30 days with an average of 17 days for frozen dural samples as compared to 5-10 days for fresh skin biopsies.

TABLE 1
Successful dural outgrowths and iPSC generation.

| Sample | CP | Sex | Age | iPSCs | Class. | PMI (Frozen) |
|---|---|---|---|---|---|---|
| ASC2S/D | Y | F | 47 | MSA | 3+ clones | 14′24″ |
| ASC7D | N | F | 79 | AD | 3+ clones | 7′40″ |
| ASC8D | N | F | 54 | Control | Contaminated | 15′40″ |
| ASC9D | N | F | 78 | AD | Insufficient # | 34′55″ |
| ASC12D | N | M | 68 | Control | Failed | 20′46″ |
| ASC14D | N | F | 78 | AD | N/A | 34′55″ |
| ASC15D | N | F | 79 | AD | N/A | 7′40″ |
| ASC19D | N | M | 60 | ALS | 2+ clones | 11′45″ |
| ASC21D | N | M | 89 | Control | Failed | 7′17″ |
| ASC22D | N | F | 54 | Control | Failed | 15′40″ |
| ASC24D | N | M | 72 | DLBD | Failed | 23′55″ |
| ASC27D | N | M | 63 | HD | 2+ clones | 14′55″ |
| ASC30D | N | F | 76 | PD | 2+ clones | 13′34″ |

Abbreviations for Table 1. For sample, S refers to skin as tissue of origin, D refers to dura mater. CP stands for cyroprotection. Class stands for classification, which includes MSA (multiple systems atrophy), AD (Alzheimer's disease), ALS (amyotrophic lateral sclerosis), DLBD (diffuse Lewy body disease), HD (Huntington's disease), and PD (Parkinson's disease). For iPSC generation, N/A (not applicable) indicates no reprogramming was attempted although the number of outgrowth cells was sufficient. PMI refers to post mortem interval, and refers to the amount of time after death before the sample was frozen in hours (′) and minutes (″).

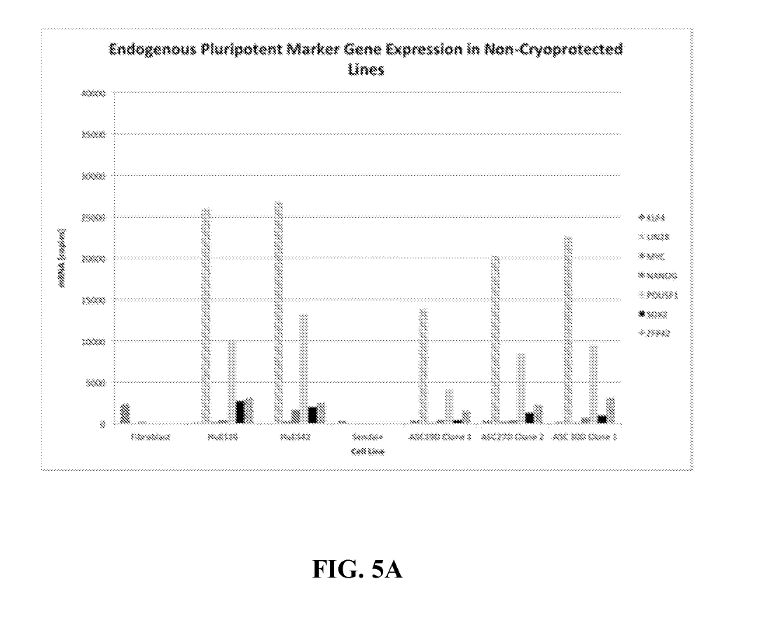

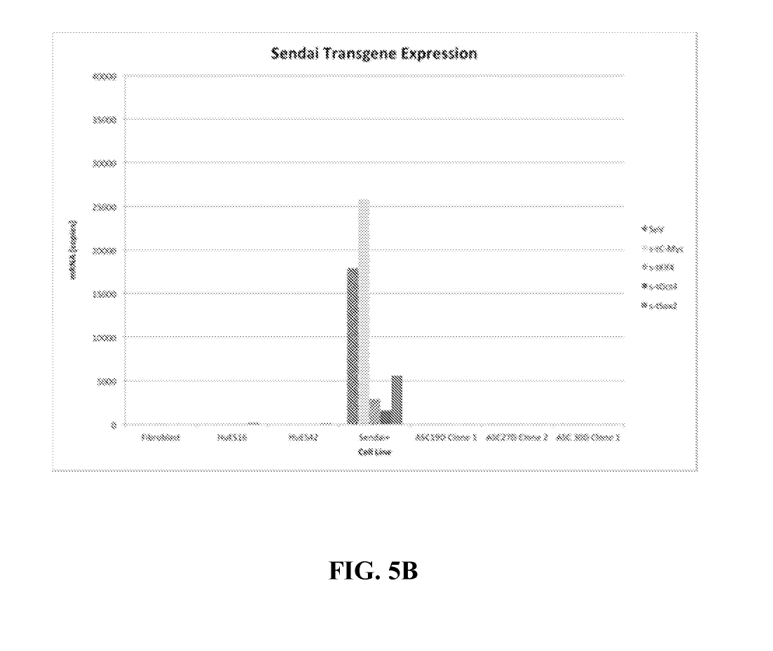

Sendai virus-mediated reprogramming was successful in three of six lines, including samples from neuropathologically-confirmed sporadic amyotrophic lateral sclerosis (ALS), Huntington's disease, and Parkinson's disease (Table 1). These tissues had been stored at −80° C. for 10-11 years. Post mortem interval before freezing ranged between 4.5 hours and 39 hours, and did not correlate with either successful outgrowth or subsequent reprogramming. Two clones from each line were manually picked and expanded, and the clone with best morphology was further characterized. Similar to what was done for ASC2D/S-MSA and ASC7D-AD reprogramming, additional clones (at least 2+ per line) were frozen in bulk for potential future use. While the reprogramming efficiency was not as high as fast growing fresh skin biopsies (20+ colonies under good conditions), it was sufficient to allow one to study a handful of clones if desired. In each case, Nanostring assays and immunostaining for endogenous stem cell markers confirmed successful reprogramming and Sendai transgene shutoff, embryoid bodies confirmed pluripotency, and iPSCs had the capacity to undergo directed differentiation into neural cells (FIGS. 3 and 5). ASC19D-ALS (amyotrophic lateral sclerosis) and ASC30D-PD (Parkinson's disease) cell lines had normal karyotypes, although multiple clones of ASC27D-HD (male Huntington's disease subject) lacked the presence of a Y chromosome. PCR amplification of the AMG gene, which can produce different amplicon sizes depending if AMG is located on the X or Y chromosome, suggests that the Y chromosome was lost in the fibroblast outgrowth culture, but is present in genomic DNA from the original dural tissue. In summary, fourteen of twenty-five (56%) dural samples produced fibroblast outgrowths, and four of nine (44%) outgrowths were successfully reprogrammed.

### Discussion

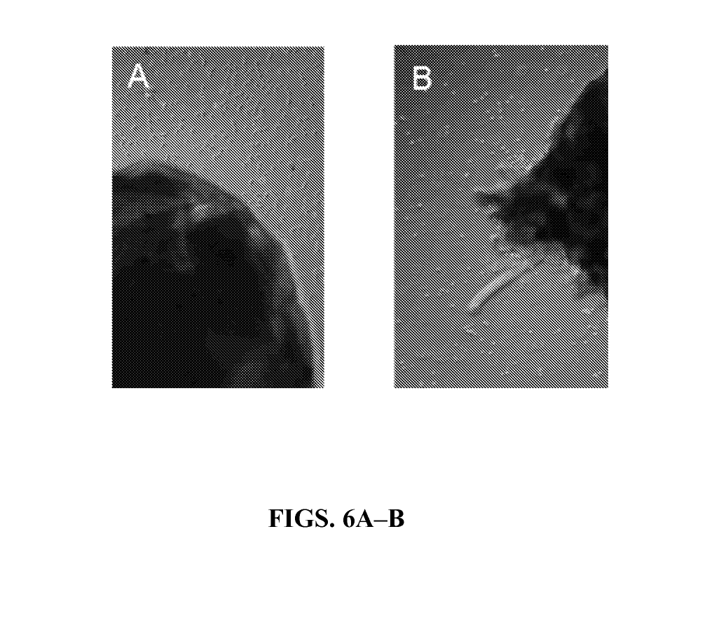

The results described in this Example demonstrate the potential to leverage existing biorepositories to support research in a powerful new way. While tissue banks in various forms have existed for over 60 years, demand for human tissue for personalized medicine and associated genetic studies, which require large biorepositories, has accelerated the rate of establishment of new facilities [11, 12]. A recent survey has identified at least 636 U.S. biobanks, 36% of which have postmortem tissue, 9% exclusively (largely brain banks) [11]. Archival specimens that were not intended for iPSC generation at the time of harvesting have the potential to be unlocked for functional studies to test mechanistic hypotheses. Preliminary results suggest other banked brain tissues, including large cerebral artery from an ALS subject (FIG. 6A), choroid plexus from a frontotemporal lobar degeneration/motor neuron disease subject (FIG. 6B), leptomeninges and the large artery associated with the choroid plexus, can also produce outgrowths both morphologically similar and distinct from fibroblasts. In addition, while many collections store solid tissues, there are other specimens within these banks that might be suitable for generating stem cell lines in the future, including blood, saliva or buccal cells, cord blood and pathological body fluids (e.g, ascites), among others [11]. The majority of biobanks focus on a single disease group, with neoplasia, neurodegeneration and HIV/AIDS being the most common, but there is a rich diversity of diseases represented in these collections that include both common and rare diseases, many of which currently have no cellular models. In the case of Alzheimer's disease, the Alzheimer's Disease Education and Referral Center (ADEAR) currently lists 27 NIA funded Alzheimer's disease centers, each containing a neuropathology core that routinely banks frozen tissue. While the total number of specimens available for generating iPSCs is difficult to estimate, the National Alzheimer's Coordinating Center (NACC) has autopsy data from ˜13,279 subjects from these centers as of Jun. 1, 2013, the majority of which have frozen tissue available.

There is a vital need for well-characterized patient material for translational research [13]. Deriving iPSCs from tissue from patients with neurodegenerative diseases with post-mortem confirmation, which remains the gold standard, is highly advantageous over utilization of lines from patients with clinical ascertainment alone in that there is certainty in the diagnosis. This approach has the additional benefit of having post-mortem brain tissue available for rapid correlation and cross validation of neuropathological and cellular findings.

### REFERENCES FOR EXAMPLE

Each of the references listed below, and all other references cited in this patent application, are hereby incorporated by reference in their entireties.
1. Bliss L A, Sams M R, Deep-Soboslay A, et al. (2012) Use of postmortem human dura mater and scalp for deriving human fibroblast cultures. PLoS ONE 7:e45282. doi: 10.1371/journal.pone.0045282
2. Hjelm B E, Rosenberg J B, Szelinger S, et al. (2011) Induction of pluripotent stem cells from autopsy donor-derived somatic cells. Neurosci Lett 502:219-224. doi: 10.1016/j.neulet.2011.07.048
3. Vonsattel J-P G, Amaya M D P, Cortes E P, et al. (2008) Twenty-first century brain banking: practical prerequisites and lessons from the past: the experience of New York Brain Bank, Taub Institute, Columbia University. Cell Tissue Bank 9:247-258. doi: 10.1007/s10561-008-9079-y
4. Vonsattel J-P G, Del Amaya M P, Keller C E (2008) Twenty-first century brain banking Processing brains for research: the Columbia University methods. Acta Neuropathologica 115:509-532. doi: 10.1007/s00401-007-0311-9
5. Kahler D J, Ahmad F S, Ritz A, et al. (2013) Improved methods for reprogramming human dermal fibroblasts using fluorescence activated cell sorting. PLoS ONE 8:e59867. doi: 10.1371/journal.pone.0059867
6. Ahmed Z, Asi Y T, Sailer A, et al. (2012) The neuropathology, pathophysiology and genetics of multiple system atrophy. Neuropathol Appl Neurobiol 38:4-24. doi: 10.1111/j.1365-2990.2011.01234.x
7. Kalluri R, Zeisberg M (2006) Fibroblasts in cancer. Nat Rev Cancer 6:392-401. doi: 10.1038/nrc1877
8. Ban H, Nishishita N, Fusaki N, et al. (2011) Efficient generation of transgene-free human induced pluripotent stem cells (iPSCs) by temperature-sensitive Sendai virus vectors. Proc Natl Acad Sci USA 108:14234-14239. doi: 10.1073/pnas.1103509108
9. Mirra S S, Heyman A, McKeel D, et al. (1991) The Consortium to Establish a Registry for Alzheimer“s Disease (CERAD). Part II. Standardization of the neuropathologic assessment of Alzheimer”s disease. Neurology 41:479-486.
10. Braak H, Braak E (1991) Neuropathological stageing of Alzheimer-related changes. Acta Neuropathologica 82:239-259.
11. Henderson G E, Cadigan R J, Edwards T P (2013) Genome Medicine 5 (1):3. doi: 10.1186/gm407.
12. Hewitt R E (2011) Biobanking: the foundation of personalized medicine. Curr Opin Oncol 23:112-119. doi: 10.1097/CCO.0b013e32834161b8
13. Founti P, Topouzis F, van Koolwijk L, et al. (2009) Biobanks and the importance of detailed phenotyping: a case study—the European Glaucoma Society GlaucoGENE project. Br J Ophthalmol 93:577-581. doi: 10.1136/bjo.2008.156273

While the foregoing invention has been described in some detail for purposes of clarity and understanding, it will be clear to one skilled in the art from a reading of this disclosure that various changes in form and detail can be made without departing from the true scope of the invention. The invention may also be further defined in terms of the following claims.

1. An in vitro model system for studying cells, or for studying the effect of one or more agents on cells, the model system comprising:
a. a cell panel comprising at least two cell populations, wherein each cell population:
i. was derived from a tissue sample obtained from a subject, wherein the tissue sample had been frozen without a cryoprotective agent, and
ii. comprises pluripotent stem cells or differentiated cells derived following reprogramming of differentiated somatic cells grown from the tissue sample, and,
b. a data panel comprising information associated with each cell population in the cell panel or each tissue sample or subject from which each cell population was derived.

2. The in vitro model system of claim 1, wherein the pluripotent stem cells are induced pluripotent stem cells.

3. The in vitro model system of claim 1, wherein the differentiated cells are derived from induced pluripotent stem cells.

4. The in vitro model system of claim 1, wherein the differentiated cells are derived by trans-differentiation of differentiated somatic cells grown from the tissue sample.

5. The in vitro model system of claim 1, wherein cells in each cell population comprise one or more exogenously-introduced reporter molecules or exogenously-introduced reprogramming factors.

6. The in vitro model system of claim 1, wherein each cell population is contained in a separate vessel.

7. The in vitro model system of claim 6, wherein the vessel is a well in a multi-well plate.

8. The in vitro model system of claim 1, wherein at least one cell population was derived from a tissue sample that was obtained from a subject post mortem.

9. The in vitro model system of claim 1, wherein one or more of the cell populations was derived from a tissue sample obtained from a test subject having a disease or disorder.

10. The in vitro model system of claim 9, wherein one or more of the cell populations was derived from a tissue sample obtained from a control subject not having the disease or disorder.

11. The in vitro model system of claim 9, wherein the disease or disorder is a neurological disease or disorder.

12. The in vitro model system of claim 11, wherein each cell population comprises differentiated neurons.

13. The in vitro model system of claim 9, wherein the neurological disease or disorder is selected from the group consisting of Alzheimer's disease (AD), Parkinson's disease (PD), amyotrophic lateral sclerosis (ALS), Huntington's disease, multiple systems atrophy (MSA), or diffuse Lewy body disease (DLBD).

14. The in vitro model system of claim 1, wherein the data panel comprises information about the subject's age, sex, race, genotype, phenotype, environment, medical history, health, family history, disease status, treatment history, histopathology, diagnosis, disease classification, disease stratification, disease grading, disease severity, or disease progression.

15. The in vitro model system of claim 1, wherein the data panel comprises information about the subject, the tissue sample, or the cell population that was obtained post-mortem.

16. A cell panel for studying cells, or for studying the effect of one or more agents on cells, the cell panel comprising at least two distinct cell populations, wherein each cell population:
a. was derived from a tissue sample obtained from a subject, wherein the tissue sample had been frozen without a cryoprotective agent, and
b. comprises pluripotent stem cells or differentiated cells derived following reprogramming of differentiated somatic cells grown from the tissue sample.

17. The cell panel of claim 16, wherein the pluripotent stem cells are induced pluripotent stem cells.

18. The cell panel of claim 16, wherein the differentiated cells are derived from induced pluripotent stem cells.

19. The cell panel of claim 16, wherein the differentiated cells are derived by trans-differentiation of differentiated somatic cells grown from the tissue sample.

20. The cell panel of claim 16, wherein cells in each cell population comprise one or more exogenously-introduced reprogramming factors, differentiation factors, or reporter molecules.

21. An induced pluripotent stem cell derived from a tissue sample obtained from a subject, wherein the tissue sample had been frozen without a cryoprotective agent.

22. The induced pluripotent stem cell of claim 21, wherein the cell comprises one or more exogenously-introduced reprogramming factors or one or more exogenously-introduced reporter molecules.

23. A differentiated cell derived from an induced pluripotent stem cell according to claim 22.

24. The differentiated cell of claim 23, wherein the cell comprises one or more exogenously-introduced differentiation factors or reporter molecules.

25. A screening method comprising:
a. contacting one or more populations of pluripotent stem cells, multipotent cells, or differentiated cells with a candidate agent, wherein each of such population of cells was derived from a tissue sample that had been frozen without a cryoprotective agent, and
b. determining an effect of the candidate agent on the population of cells.

26. The screening method of claim 25, wherein at least one cell population was derived from a tissue sample that was obtained from a subject post mortem.

27. The screening method of claim 25, wherein one or more of the populations of cells was derived from a tissue sample obtained from a test subject having a disease or disorder.

28. The screening method of claim 25, wherein one or more of the populations of cells was derived from a tissue sample obtained from a control subject not having the disease or disorder.

29. The screening method of claim 25, wherein cells in each population of cells comprise one or more exogenously-introduced reporter molecules.

30. The screening method of claim 25, where the step of determining the effect of the candidate agent on the population of cells comprises assessing the agent's activity, efficacy, potency, safety, toxicity, or teratogenicity.

31. A method of generating induced pluripotent stem cells comprising:
a. obtaining a tissue sample from a subject, wherein the tissue sample had been frozen without a cryoprotective agent,
b. culturing cells from the tissue sample, and
c. contacting the cells cultured in step b. with one or more reprogramming factors to generate induced pluripotent stem cells.

32. The method of claim 31, further comprising contacting the induced pluripotent stem cells of step c. with one or more differentiation factors to generate differentiated cells.
